(19) United States
(12) Patent Application Publication
(10) Pub. No.: US 2012/0101261 A1
(43) Pub. Date: Apr. 26, 2012

(54) USE OF MULTI-KINASE INHIBITORS IN THE TREATMENT OF VASCULAR HYPERPERMEABILITY

(75) Inventors: Alessandro Massimo Gianni, Milano (IT); Carmelo Carlo-Stella, Milano (IT)

(73) Assignee: Fondazione IRCCS Istituto Nazionale Dei Tumori, Milano (IT)

(21) Appl. No.: 13/263,402

(22) PCT Filed: Apr. 15, 2009
(86) PCT No.: PCT/IB2009/051566

§ 371 Date: Nov. 29, 2011

(51) Int. Cl. C07K 16/00; C07D 213/78; C07D 403/06; C07D 401/06

(52) U.S. Cl. 530/387.3; 544/238; 546/298; 548/468

(57) ABSTRACT

A multi-kinase inhibitor, in particular Sorafenib, is used for the preparation of a pharmaceutical composition in the treatment of a variety of pathological conditions involving vascular hyperpermeability in order to reduce vascular hyperpermeability.

### BACKGROUND

1. Field

The present invention relates to the use of multi-kinase inhibitors, in particular of sorafenib in the treatment of a variety of pathological conditions involving vascular hyperpermeability.

In particular, the present invention relates to the use of multi-kinase inhibitors, in particular of sorafenib in the treatment of limphedema, cerebral edema, burns, retinal edema, sepsis, cardiovascular diseases (e.g. heart failure), ascites secondary to portal hypertension.

2. Background Art

### Vascular Hyperpermeability

Several different and potentially severe pathological conditions critically involve an increase of vascular permeability. A non-exhaustive list of such pathological conditions may include:
lymphedema following surgical dissection of, and/or radiotherapy on lymphnodes;
cerebral edema (neoplastic, vascular);
burns;
retinal edema;
sepsis;
cardiovascular diseases (e.g., heart failure);
ascites secondary to portal hypertension.

These pathological conditions are currently treated by means of different therapeutic options. By way of example, acquired lymphedema following surgical lymphadenectomy and/or radiotherapy is a frequent and clinically relevant event in patients with solid tumors. Following dissection of regional lymphnodes, 20 to 25% of breast cancer patients and 40 to 50% of melanoma patients develop lymphedema, whereby the incidence of lymphedema is significantly increased in patients receiving post-operative locoregional radiotherapy.

Currently, there is no effective therapeutic option for lymphedema, whereby the available treatments are of limited efficacy and include elastic compression garments associated with lymphatic drainage.

Cerebral edema, irrespective of its neoplastic or vascular origin, is currently treated through, e.g. osmotherapy, diuretics and corticosteroids.

Furthermore, the treatment of burns is carried out by means of hydration, antibiotics, analgesics and skin grafting.

Yet, retinal edema is treated through corticosterois.

Again, sepsis is treated by means of antibiotics, recombinant human activated protein C and corticosteroids.

Moreover, cardiac failures involve a series of different therapies such as ACE inhibitors, β-blockers, aldosterone antagonists, diuretics, angiotensin II receptor antagonist therapy, positive inotropes and, in the case of infartual edema, corticosteroids.

On their turn, ascites with portal hypertension are currently treated through salt restriction, diuretics and paracentesis.

As mentioned above, these pathological conditions involve an increase of vascular permeability that is caused by the activation of the proangiogenic vascular endothelial growth factor receptors (VEGFR1)-1, VEGFR-2, VEGFR-3, and platelet-derived growth factor receptor PDGFR (Bates D O, Harper S J. Regulation of vascular permeability by vascular endothelial growth factors. Vascul Pharmacol. 2002; 39:225-237).

### Multikinase Inhibitors

Sorafenib (Nexavar, BAY43-9006) is an oral multikinase inhibitor with antiproliferative and antiangiogenic properties that is generally known and used for treating cancer (Wilhelm S, Carter C, Lynch M, et al. Discovery and development of sorafenib: a multikinase inhibitor for treating cancer. Nat Rev Drug Discov. 2006; 5:835-844).

It is known that sorafenib blocks tumor cell proliferation by inhibiting the RAF/MEK/ERK pathway in several cell lines from from both hematopoietic malignancies and solid tumors. Additionally, sorafenib inhibits the receptor tyrosine kinases c-kit, Flt3, RET, and the antiapoptotic protein Mcl-1, a member of the Bcl-2 family (Meng X W, Lee S H, Dai H, et al. Mcl-1 as a buffer for proapoptotic Bcl-2 family members during TRAIL-induced apoptosis: a mechanistic basis for sorafenib (Bay 43-9006)-induced TRAIL sensitization. J Biol Chem. 2007; 282:29831-29846, and Rahmani M, Davis E M, Bauer C, Dent P, Grant S. Apoptosis induced by the kinase inhibitor BAY 43-9006 in human leukemia cells involves down-regulation of Mcl-1 through inhibition of translation. J Biol Chem. 2005; 280:35217-35227).

Moreover, sorafenib inhibits tumor growth in a variety of preclinical models of human melanoma, renal, colon, pancreatic, hepatocellular, thyroid, and ovarian carcinomas and NSCLC (Wilhelm S M, Carter C, Tang L, et al. BAY 43-9006 exhibits broad spectrum oral antitumor activity and targets the RAF/MEK/ERK pathway and receptor tyrosine kinases involved in tumor progression and angiogenesis. Cancer Res. 2004; 64:7099-7109).

Furthermore, sorafenib produced partial tumor regressions in mice bearing PLC/PRF/5 HCC and induced substantial tumor regression in a breast cancer model harboring B-Raf and K-Ras oncogenic mutations (Liu L, Cao Y, Chen C, et al. Sorafenib blocks the RAF/MEK/ERK pathway, inhibits tumor angiogenesis, and induces tumor cell apoptosis in hepatocellular carcinoma model PLC/PRF/5. Cancer Research. 2006; 66:11851-11858).

Sorafenib is approved by the U.S. Food and Drug Administration for the treatment of patients with advanced renal cell carcinoma (RCC) and those with unresectable hepatocellular carcinoma (HCC) (Escudier B, Eisen T, Stadler W M, et al. Sorafenib in advanced clear-cell renal-cell carcinoma. N Engl J Med. 2007; 356:125-134 and Llovet J M, Ricci S, Mazzaferro V, et al. Sorafenib in advanced hepatocellular carcinoma. N Engl J Med. 2008; 359:378-390).

In addition to targeting RAF serine/thronine kinases, sorafenib is also known as a potent inhibiting factor of the proangiogenic vascular endothelial growth factor receptors (VEGFR1)-1, VEGFR-2, VEGFR-3, and of the platelet-derived growth factor receptor (PDGFR) (Wilhelm S M, Adnane L, Newell P, Villanueva A, Llovet J M, Lynch M. Preclinical overview of sorafenib, a multikinase inhibitor that targets both Raf and VEGF and PDGF receptor tyrosine kinase signaling. Molecular Cancer Therapeutics. 2008; 7:3129-3140).

Sorafenib is also approved by the European Medicines Agency for the treatment of patients with HCC and patients with advanced RCC with whom prior IFN-alfa or interleukin-2-based therapy had failed or those considered to be unsuitable for such therapy.

In all known uses of sorafenib the recommended daily dosing is 800 mg.

Yet, sorafenib is undergoing phase II/III clinical evaluation in a wide variety of other solid as well as hematopooietic tumors, including melanoma, non-small cell lung cancer, non-Hodgkin lymphoma.

Currently there is no approved indication for the use of sorafenib in non-neoplastic diseases. However, clinical development of sorafenib for treatment of pulmonary hypertension can be anticipated based on a recent publication showing that sorafenib prevents pulmonary remodeling and improves cardiac and pulmonary function in experimental pulmonary hypertension due to inhibition of the Raf kinase pathway (Klein M, Schermuly R T, Ellinghaus P, et al. Combined tyrosine and serine/threonine kinase inhibition by sorafenib prevents progression of experimental pulmonary hypertension and myocardial remodeling. Circulation. 2008; 118:2081-2090).

Several patent publications disclose the use of sorafenib in the treatment of cancer. Among these we may cite EP-A-1954272, EP-A-1568589, U.S. Pat. No.7,351,834, U.S. Pat. No. 2,803,825A1, WO-A-27053573, WO-A-27059155.

Other multikinase inhibitors showing properties similar to-sorafenib have recently been developed. Among these we can cite bevacizumab, sunitinib and vatalanib, this list being not exhaustive.

Bevacizumab, in combination with intravenous 5-fluorouracil-based (5-FU) chemotherapy, is approved for first- or second-line treatment of patients with metastatic carcinoma of the colon or rectum.

Bevacizumab, in combination with carboplatin and paclitaxel, is approved for the first-line treatment of patients with unresectable, locally advanced, recurrent or metastatic non-squamous non-small cell lung cancer (NSCLC).

Bevacizumab, in combination with paclitaxel, is approved for the treatment of patients who have not received chemotherapy for metastatic HER2-negative breast cancer.

Sunitinib is indicated for the treatment of gastrointestinal stromal tumor after disease progression on or intolerance to imatinib mesylate.

Sunitinib is indicated for the treatment of advanced renal cell carcinoma.

Finally, vatanalib is Under development and there is no current FDA-approved indication.

### SUMMARY OF THE INVENTION

The present invention relates to the use of multi-kinase inhibitors such as sorafenib, bevacizumab, sunitinib, vatalanib and others in the treatment of a variety of pathological conditions involving vascular hyperpermeability, in particular in the treatment of limphedema, cerebral edema, burns, retinal edema, sepsis, cardiovascular diseases (e.g. heart failure), ascites secondary to portal hypertension.

Such use is described in the main claim. The dependent claims outline further advantageous way of using multi-kinase inhibitors.

The use of multi-kinase inhibitors for the treatment of pathologies involving vascular hyper permeability represents the first effective and well tolerated pharmacological treatment for this frequent and debilitating progressive condition with no known cure.

### DRAWINGS

Other features and advantages of the invention will become apparent by reading the following description of some forms of embodiment of the invention, given as non-limiting examples and with the help of the figures illustrated in the attached drawings, in which:

FIG. 1 shows the results of an experiment carried out on mice tails with an induced lymphedema, and respectively treated or non-treated with a multi-kinase inhibitor;

FIG. 2 shows the results of an experiment relative to the presence of vascular hyperpermeability in tails of mice which were respectively treated or non-treated with a multi-kinase inhibitor.

### DESCRIPTION OF SOME FORMS OF EMBODIMENT OF THE INVENTION

Recently, the Applicants observed a complete regression of a severe bilateral leg lymphedema in a patient with Hodgkin lymphoma receiving multi-kinase inhibitor sorafenib at 800 mg/die in the context of a phase II clinical trial. In this patient, lymphedema was due a compressive lymphatic obstruction related to post-radiotherapy sequelae.

As a mechanistic explanation of this observation, the present Applicants hypothesized that sorafenib might have inhibited vascular permeability by suppressing VEGFRs, ultimately surprisingly inducing the reduction of lymphedema. In fact one major activity of VEGF, the ligand of VEGFRs, is an increase in vascular permeability, and this protein was also known as Vascular Permeability Factor (VPF).

To test this hypothesis, the Applicants initially evaluated the effect of multi-kinase inhibitor sorafenib in a mouse tail model of lymphedema, and next investigated whether sorafenib might interfere with vascular permeability using the Miles vascular permeability assay.

### EXPERIMENT 1

### Mouse Tail Model of Lymphedema

Six- to eight-wk-old female C57BL/6 mice with body weight of 20 to 25 g, were purchased from Charles River (Milano, Italy, EU). Mice were housed under standard laboratory conditions according to the Applicant's institutional guidelines. Animal experiments were performed according to the Italian laws (Law Decree 116/92 and following additions), which enforce the EU 86/109 Directive, and were approved by the institutional Ethical Committee for Animal Experimentation.

To create lymphedema, a circumferential incision was made through the dermis close to the tail base to sever the dermal lymphatic vessels. The edges of this incision were then pushed apart, thereby severing the deeper draining lymphatics, preventing superficial bleeding, and creating a 2-3 mm gap to delay wound closure. Care was taken to maintain the integrity of the major underlying blood vessels and tendons so that the tail distal to the incision did not become necrotic.

Five days following circumferential incision, mice showed distal tail lymphedema and were randomly assigned to receive control vehicle or sorafenib (60 mg/kg/die) from days 5 to 9 and 12 to 16. Sorafenib dosing used in mice experiments is roughly equivalent to a 300 mg daily dosing in humans.

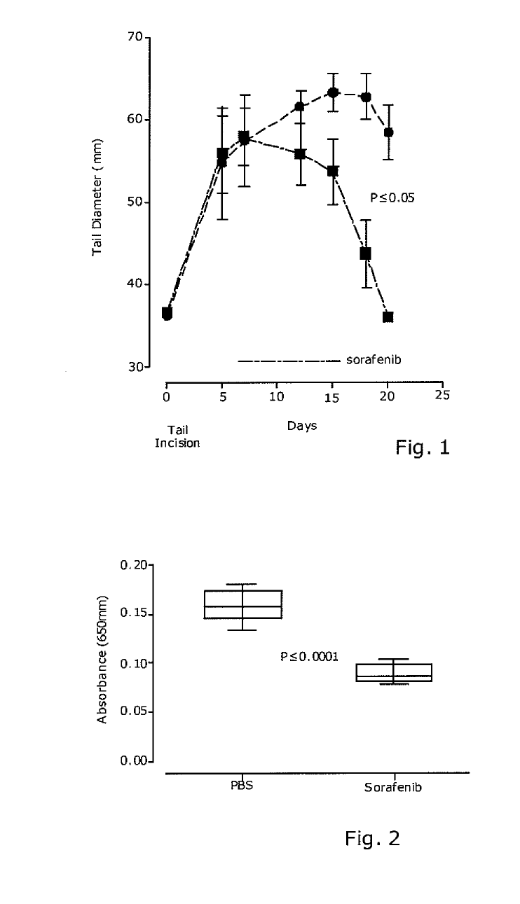

On day 5 following circumferential incision, the mean (±SEM) tail diameter was significantly increased as compared to baseline values (55±7 vs 36±1, P≦0.002) due to the consistent occurrence of an ingravescent tail lymphedema (FIG. 1). Mice were than randomly assigned to receive two cycles of sorafenib (60 mg/kg/die) from days 5 to 9 and 12 to 16 or control vehicle. Control mice displayed a progressive increase of lymphedema peaking on day 15 when a mean tail diameter of 63±3 mm was recorded. In striking contrast (see FIG. 1), sorafenib-treated mice experienced a lymphedema peaking on day 7 when a 58±4 mm tail diameter was recorded which was followed by a progressive and quick decline of tail volume resulting in a complete resolution of lymphedema on day 20 when control mice still showed relevant tail lymphedema (36±1 vs 58±3, P≦0.0003).

### EXPERIMENT 2

### Miles Vascular Permeability Assay

Since a significantly reduced edema formation in sorafenib-treated mice was found, the Applicants next investigated whether multi-kinase inhibitor sorafenib might reduce vascular hyperpermeability. A Miles vascular permeability assay using intrasplenic injection of the blue dye Evans blue was perfomed in untreated and sorafenib-treated mice bearing a surgical-induced tail lymphedema.

Six- to eight-wk-old female C57BL/6 mice with body weight of 20 to 25 g, were used in this experiment. Tail lymphedema was generated by a circumferential incision through the dermis close to the tail base, as described above. Five days following circumferential incision, mice showed distal tail lymphedema and were randomly assigned to receive control vehicle or sorafenib (60 mg/kg/die) from days 5 to 9 and 12 to 16. On day 16, mice received the last dose of sorafenib and 2 hrs later were injected through the spleen with 0.1 ml of 1% Evan's blue in PBS. After 2 h, the mice were exsanguinated under anesthesia, and were perfused with heparin in PBS until lungs and livers were blanched. The distal portion of the tail was then removed and placed in formamide at 37° C. overnight to extract Evan's blue dye. The Evan's blue in the tail was quantified by measuring the absorbance of the supernatants at 650 nm with a spectrophotometer.

As shown in FIG. 2, vascular hyperpermeability was detected in mouse tail, as evidenced by the increased leakage of Evans blue in untreated mice with tail edema. Spectrophotometric measurements of the amount of extravasated Evans blue revealed a significant 2-fold reduction of vascular permeability in sorafenib-treate mice as compared with levels observed in untreated mice [mean (±SEM) OD₆₂₀: 0.09±0.009 vs 0.16±0.01, P≦0.0001]. The reduced extravasation of Evan's blue indicates that sorafenib has the capacity of significantly reducing vascular permeability in vivo.

### CLINICAL STUDY

To further evaluate in a clinical setting the anti-lymphedema activity of multi-kinase inhibitor sorafenib, the Applicants performed a pilot study on a compassionate need basis to evaluate the toxicity and the anti-lymphedema effects of multi-kinase inhibitor sorafenib in consenting breast cancer patients with acquired arm lymphedema occurring following surgical dissection of, and/or radiotherapy on axillary lymphnodes. No other treatment options of proven efficacy was available for these patients.

Patients were administered with oral sorafenib at a dose of 200 mg daily.

Therapy was given for a maximum of 8 weeks or until lymphedema progression or appearance of clinical significant toxicity probably related to multi-kinase inhibitor sorafenib.

Within a two-month period of time ten breast cancer patients with acquired lymphedema of the arm were treated with multi-kinase inhibitor sorafenib.

The median time from onset of lymphedema was 24 months (range, 6 to 48). Overall, sorafenib was well tolerated and all patients received the planned treatment with no dose reduction or treatment discontinuation due to occurrence of any type of toxicity of any grade.

At the end of treatment, the efficacy of multi-kinase inhibitor sorafenib was evaluated as percentage reduction of total arm circumference as compared with pre-treatment values. The median reduction of total arm circumference was 60% (range, 30 to 100). Lymphedema reduction was associated with a 5-10% reduction of body weight.

### CONCLUSIONS

Preclinical and clinical data reported herein strongly support a potent efficacy of multi-kinase inhibitor sorafenib in the treatment of acquired lymphedema. The effect of multi-kinase inhibitor sorafenib has been detected at the preclinical level in the mouse tail model of lymphedema and has been confirmed in the clinical setting of iatrogenic lymphedema occurring in breast cancer patients following surgical dissection of, and/or radiotherapy on axillary lymphnodes. Sorafenib was administered using a 200 mg once-daily schedule which represents ¼ of the conventional antitumor schedule of this drug. This prevented dangerous side effects and showed that multi-kinase inhibitor sorafenib may very well find an indication in the treatment of a variety of pathological conditions involving vascular hyperpermeability in order to reduce said vascular hyperpermeability.

The activation of VEGFRs, what induces vascular hyperpermeability, has therefore been mediated by a strong anti-edema activity caused by a treatment with multi-kinase inhibitor sorafenib. At the preclinical level, the Miles vascular permeability assay indeed suggests that multi-kinase inhibitor sorafenib has the capacity of significantly reducing vascular permeability in vivo.

Since the inhibition of VEGFRs is a property shared by several multi-kinase inhibitors such as bevacizumab, sunitinib, vatalanib and others, these multi-kinase inhibitors can efficiently be used in order to reduce vascular hyperpermeability in a series of pathological conditions like limphedema, cerebral edema, burns, retinal edema, sepsis, cardiovascular diseases (e.g. heart failure), ascites secondary to portal hypertension.

1-12. (canceled)

13. Use of a multi-kinase inhibitor for the preparation of a pharmaceutical composition in the treatment of a variety of pathological conditions involving vascular hyperpermeability in order to reduce vascular hyperpermeability.

14. Use according to claim 13, wherein said multi-kinase inhibitor is sorafenib.

15. Use according to claim 13, wherein said multi-kinase inhibitor is bevacizumab.

16. Use according to claim 13, wherein said multi-kinase inhibitor is sunitinib.

17. Use according to claim 13, wherein said multi-kinase inhibitor is vatalanib.

18. Use according to claim 13, wherein the pathological condition is lymphedema.

19. Use according to claim 13, wherein the pathological condition is cerebral edema.

20. Use according to claim 13, wherein the pathological condition is a burn.

21. Use according to claim 13, wherein the pathological condition is retinal edema.

22. Use according to claim 13, wherein the pathological condition is sepsis.

23. Use according to claim 13, wherein the pathological condition is a cardiovascular disease, e.g. a heart failure.

24. Use according to claim 13, wherein the pathological condition is an ascite secondary to portal hypertension.
